SEQUENCE LISTING (3 sequences)

SEQ ID NO: 1 - DNA, 24 bp, Artificial Sequence
  Synthetic Primer
atcaacaaagcccagaagcagaaa

SEQ ID NO: 2 - DNA, 29 bp, Artificial Sequence
  Synthetic Primer
aatattcccatcagtatcactattttatg

SEQ ID NO: 3 - DNA, 31 bp, Artificial Sequence
  Synthetic Probe
cccttgtagttactgaatctgacaccgaatt

(19) United States
(12) Patent Application Publication
(10) Pub. No.: US 2020/0340038 A1
(43) Pub. Date: Oct. 29, 2020

(54) Portable System and Process Thereof to Rapidly Filter, Concentrate, and Detect Waterborne Pathogens

(71) Applicant: Nephros Inc., South Orange, NJ (US)

(72) Inventors: Daron Evans, Woodside, CA (US); Kimothy Smith, Incline Village, NV (US)

(21) Appl. No.: 16/854,694

(22) Filed: Apr. 21, 2020

Related U.S. Application Data
(60) Provisional application No. 62/838,524, filed on Apr. 25, 2019.

(51) Int. Cl. C12Q 1/686; C02F 1/00; B01D 63/02

(52) U.S. Cl. CPC C12Q 1/686; B01D 63/02; C02F 1/001

(57) ABSTRACT

Disclosed herein is a high efficiency filtration device and a method thereof for analysis of a fluid sample to detect the presence of target pathogens or indicator microorganisms. Also disclosed herein is a kit to detect the presence of target pathogens or indicator microorganisms, comprising the disclosed filtration device and a molecular detection device. Also disclosed herein is a hardware mobile electronic device and a software application that analyze a fluid sample to detect the presence of target pathogens or indicator microorganisms and wirelessly transmits such information to users.

### CROSS-REFERENCE TO RELATED PATENT APPLICATIONS

This application is based on and claims priority to U.S. Provisional Patent Application 62/838,524, filed Apr. 25, 2019, the entire contents of which is incorporated by reference herein as if expressly set forth in its respective entirety herein.

### TECHNICAL FIELD

The present invention is directed to a system and process for detecting waterborne pathogens and particularly, to a portable apparatus and process for rapidly filtering, concentrating and detecting waterborne pathogens, such as bacteria, fungi and viruses, and more particularly, to a high efficiency filtration system and a method thereof for analysis of a fluid sample to detect the presence of target pathogens or indicator microorganisms. Also disclosed herein is a kit to detect the presence of target pathogens or indicator microorganisms, comprising the disclosed filtration device and a molecular detection device. Also disclosed herein is a hardware mobile electronic device and a software application that analyze a fluid sample to detect the presence of target pathogens or indicator microorganisms and wirelessly transmits such information to users and provides long term digital storage of that information on remote access computer servers.

### BACKGROUND

Many waterborne pathogens cause infections and human disease via ingestion of contaminated water. Various human parasites and pathogens are transmitted in this way, including protozoa, virus and bacteria, transmitted via human fecal contamination of water used for drinking, bathing, recreation, harvesting of shellfish, or washing/preparation of foods. Warm stationary domestic water found in air conditioner cooling towers, inadequately chlorinated swimming pools and spas, hot water heaters, respiratory therapy equipment and shower heads, have been identified as sources of infectious outbreaks. The need for and adequacy of water purification and the safety of natural waters is paramount, and sources of such water reservoir are routinely monitored by standard microbiological tests for infectious flora.

Detection and analysis of waterborne pathogens can be employed for effective prevention of infectious outbreaks. The identity of an infectious species in a sample can be ascertained by comparing the nucleic acid present in the sample to the known nucleic acid sequence. Before making this comparison, however, the nucleic acids must be extracted from the sample, amplified, and then detected. Typically, these steps take place over the course of hours, or days in a laboratory. For example, amplification usually involves the polymerase chain reaction (PCR) as described in U.S. Pat. Nos. 4,683,202 and 4,683,195. To prepare for the nucleic acids amplification using conventional PCR, the biological sample containing nucleic acids must be treated with lyse solution and incubated for at least hours.

Traditional methods and devices for extraction, amplification, and detection of nucleic acids are not typically designed to be performed in a mobile or field setting outside a specialized lab infrastructure. Extraction and amplification alone takes hours if not days, depending on the type of organism, the length of the nucleic acid strand, and the number of cycles. Absent tightly controlled test setting, contaminants can interfere with the nucleic acid polymerase enzymes used in replication, reducing the efficiency and fidelity of the amplification process. Therefore, there is an unmet need for a rapid, accurate and portable device and for detecting, quantifying and identifying target nucleic acids.

Thus, the current state of the art requires samples to be collected from remote locations and delivered to a centralized laboratory facility for detection of waterborne pathogens. This process typically takes up to two weeks to complete and cannot be implemented in a rapid, portable procedure on-site at the remote location.

### SUMMARY

In one aspect, a method for rapidly isolating and detecting waterborne pathogens within a liquid sample is provided and comprises the steps of:
filtering the liquid sample using a portable filtration device so that the waterborne pathogens forms a concentrated filtrate that comprises the waterborne pathogens and is contained within the portable filtration device;
introducing a lysing agent into the portable filtration device resulting in lysing of the waterborne pathogens and formation of a lysed waterborne pathogen solution that is contained within the portable filtration device;
removing an amount of the lysed waterborne pathogen solution from the portable filtration device; and
introducing at least a portion of the removed amount of lysed waterborne pathogen solution into a molecular detection device that is configured to detect whether target waterborne pathogens are present in the liquid sample.

In another aspect, a method for rapidly isolating and detecting waterborne pathogens within a liquid sample is provided and comprises the steps of:
filtering the liquid sample using a portable filtration device so that the waterborne pathogens forms a filtrate on a surface of a filter of the portable filtration device and purified liquid that results from the liquid sample being conducted across the filter is discharged from the portable filtration device;
using a fluid delivery device to introduce a lysing agent into the portable filtration device resulting in lysing of the waterborne pathogens and formation of a lysed waterborne pathogen solution;
using the fluid delivery device to remove an amount of the lysed waterborne pathogen solution; and
introducing at least a portion of the removed amount of lysed waterborne pathogen solution into a molecular detection device that is configured to detect whether target waterborne pathogens are present in the liquid sample.

In another aspect, a kit is provided for rapidly detecting waterborne pathogens and includes a portable filtration device including a first port for receiving a liquid sample to be tested. The portable filtration device also includes a second port for venting air and a third port for discharging purified liquid. The portable filtration device further includes a plurality of hollow semi-permeable fibers for filtering the liquid sample and generating a filtrate within lumens of the hollow semi-permeable fibers. The filtrate contains the waterborne pathogens in a concentrated form. The kit also includes a syringe configured for being sealingly mated to the first port and configured to deliver a lysing agent into lumens of the plurality of hollow semi-permeable fibers for lysing of the waterborne pathogens and formation of a lysed waterborne pathogen solution. The syringe is further configured for removing the lysed waterborne pathogen solution from within the lumens. A molecular detection device is provided for analyzing the lysed waterborne pathogen solution and detecting whether target waterborne pathogens are present in the liquid sample.

### BRIEF DESCRIPTION OF THE DRAWING FIGURES

For the purpose of illustrating the invention, there are depicted in drawings certain embodiments of the invention. However, the invention is not limited to the precise arrangements and instrumentalities of the embodiments depicted in the drawings.

The patent or application file contains at least one drawing executed in color. Copies of this patent or patent application publication with color drawing(s) will be provided by the Office upon request and payment of the necessary fee.

FIG. 1 is a schematic illustrating a portable system and process thereof to rapidly filter, concentrate and detect waterborne pathogens;

FIG. 2 is a schematic illustrating a portable system and process thereof to rapidly filter, concentrate and detect waterborne pathogens;

FIG. 3 is a cross-sectional view of a portable filtration device for use in the system;

FIG. 4 is a schematic of a portable filtration device according to another embodiment;

FIG. 5 is a schematic of a portable filtration device according to another embodiment;

FIG. 6 is a schematic of a portable filtration device and a device for delivering liquid to the portable filtration device;

FIG. 7 is a perspective view of a kit including the system of the present invention;

FIG. 8 is a perspective view of a sealed package including components of the system;

FIG. 9 is a perspective view of another kit including the system of the present invention;

FIG. 10 is a perspective view of another sealed package including components of the system; and

FIG. 11 is a perspective view of a hard shell case for containing the system.

FIG. 12 shows the results of multi-pathogen PCR performed on water samples. FIG. 12A is a table of the results of PCR on unfiltered and unconcentrated water. FIG. 12B is a graph of the results of PCR on unfiltered and unconcentrated water. FIG. 12C is a table of the results of PCR on filtered and concentrated water. FIG. 12D is a graph of the result of PCR on filtered and concentrated water.

FIG. 13 shows the results of additional multi-pathogen PCR performed on water samples. FIG. 13A is a table of the results of PCR on unfiltered and unconcentrated water. FIG. 13B is a graph of the results of PCR on unfiltered and unconcentrated water. FIG. 13C is a table of the results of PCR on filtered and concentrated water. FIG. 13D is a graph of the results of PCR on filtered and concentrated water.

FIG. 14 shows the results of gram-negative pathogen targeted quantitative PCR performed on water with low level dilutions of E. coli. FIG. 14A is a table of the results. FIG. 14B is a graph of the results.

### DETAILED DESCRIPTION OF CERTAIN EMBODIMENTS

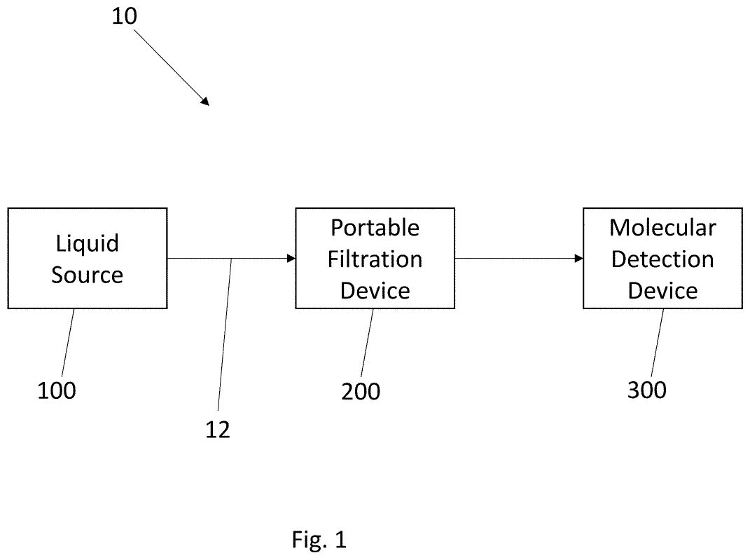

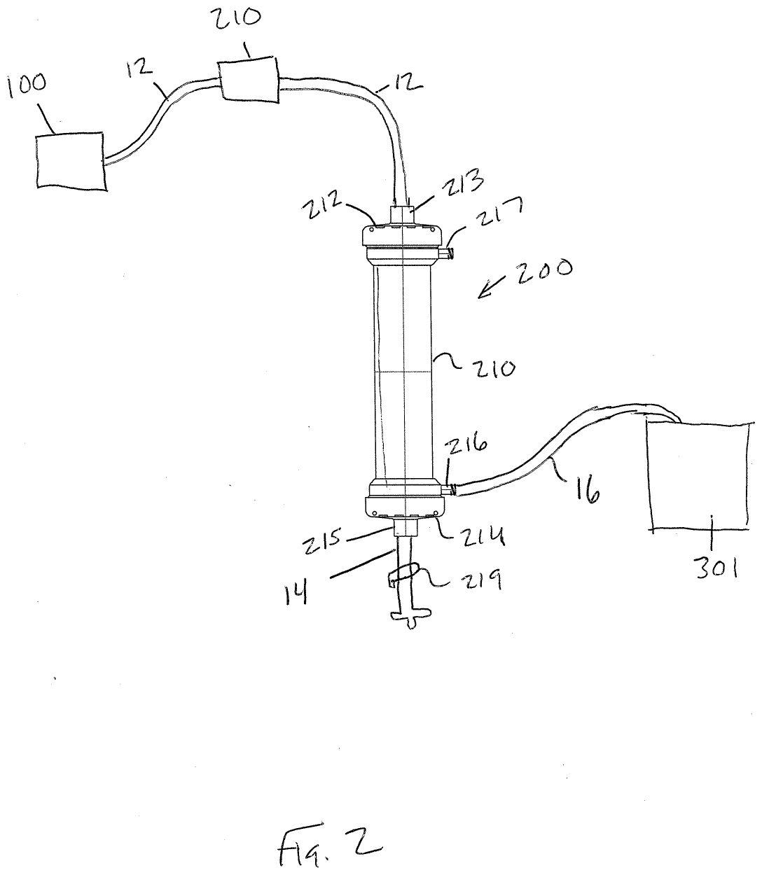

As described herein, the present invention is generally and broadly directed to a rapid, portable system and process to filter, concentrate, and detect bacteria, fungi, and viruses in liquids (liquid samples) using molecular biological methods, such as molecular detection devices, etc. For example, FIG. 1 is a schematic illustrating exemplary details of the present invention and more particularly, FIG. 1 illustrates a portable system 10 that is configured to rapidly filter, concentrate and detect waterborne pathogens. The system 10 includes a number of components that are portable in nature and can be contained and packaged as a kit, as described herein. For example, the system 100 is configured to collect a liquid sample from a liquid source 100 and deliver it to a portable filtration device 200 that is configured to rapidly filter and concentrate the target pathogens and then finally the concentrated filtrate is removed from the filtration device 200 and delivered to a molecular detection device 300 for analyzing the liquid sample. The details and operation of the system 10, as well as all of the components thereof, are set forth below.

### Definitions

Unless defined otherwise, all technical and scientific terms used herein have the same meaning as commonly understood by those of ordinary skill in the art to which this disclosure belongs. Although any methods and materials similar or equivalent to those described herein can be used in the practice or testing of the present disclosure, the preferred methods and materials are described. For purposes of the present disclosure, the following terms are defined below.

The terms “target pathogen” or “target nucleic acid” or grammatical equivalents herein are meant any biomolecule or compound to be detected. Suitable biomolecules include, but are not limited to, proteins (including enzymes, immunoglobulins and glycoproteins), nucleic acids, lipids, lectins, carbohydrates, hormones, whole cells (including prokaryotic (such as pathogenic bacteria) and eukaryotic cells, including mammalian tumor cells), viruses, spores, etc.

The term “sample” in the present specification and claims are used in their broadest sense. On the one hand it is meant to include a specimen or culture. On the other hand, it is meant to include both biological and environmental samples. In addition, a “sample” may or may not contain nucleic acid.

The term “nucleic acid” or grammatical equivalents herein means at least two nucleotides covalently linked together. A nucleic acid of the present disclosure will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977). Other analog nucleic acids include those with bicyclic structures including locked nucleic acids, Koshkin et al., J. Am. Chem. Soc. 120:13252-3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook.

The nucleic acids may be single stranded or double stranded, as specified, or contain portions, of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH).

The term “probe” refers to an oligonucleotide (i.e., a sequence of nucleotides), whether occurring naturally as in a purified restriction digest or produced synthetically, which is capable of hybridizing to another oligonucleotide of interest. Probes are useful in the detection, identification, quantification and isolation of particular gene sequences.

As used herein, the term “portable” refers to a system or device or mobile device that can be easily carried or conveyed by hand by a person. As used herein, the term “mobile device” refers to a small portable device, typically having a display screen with touch input and/or a miniature keyboard, including for example a smart phone, tablet, laptop or other portable medical device.

The terms “application” or “software” are used herein in a generic sense to refer to any type of computer code or set of computer-executable instructions that can be employed to program a computer or other processor to implement various aspects of the present disclosure as discussed above.

Target Pathogens As described herein, the portable system 10 is configured to collect and detect any number of different pathogens that may be found in a liquid sample (i.e., “target pathogens”). The following table is merely a list of exemplary target pathogens and is not to be construed as an exhaustive list and is not limiting of the scope of the present invention.

The following table lists exemplary species, number of isolates and sources of the organisms that can be used in the disclosed system. In addition to type cultures and lab-adapted strains, the present disclosure can use environmental isolates as well.

TABLE 1
Exemplary and Potential Target Pathogens
Target Pathogen

| | |
|---|---|
| 1. | Eschericia coli |
| 2. | Pseudomonas aeruginosa |
| 3. | Legionella spp. |
| 4. | Legionella pnemophila (all) |
| 5. | Legionella pnemophila Serogroup 1 |
| 6. | Legionella pneumophila Serogroup 2 |
| 7. | Legionella pneumophila Serogroup 4 |
| 8. | Legionella pneumophila Serogroup 5 |
| 9. | Legionella pneumophila Serogroup 6 |
| 10. | Legionella pneumophila Serogroup 9 |
| 11. | Legionella pneumophila Serogroup 10 |
| 12. | Legionella pneumophila Serogroup 12 |
| 13. | Legionella micdadei |
| 14. | Legionella bozemanii |
| 15. | Legionella gormanii |
| 16. | Camylobacter jejuni |
| 17. | Giardia intestinalis |
| 18. | Shigella spp. (includes S. sonnei) |
| 19. | Salmonella enterica |
| 20. | Eschericia coli O157:H7 |
| 21. | Eschericia coli K12 |
| 22. | Norovirus GI |
| 23. | Norovirus (all) |
| 24. | Rotavirus |
| 25. | Cryptosporidium (C. hominis, C. parvum) |
| 26. | Helicobacter pylori |
| 27. | Mycobacterium avium |
| 28. | Mycobacterium spp. |
| 29. | Burholderia cepacia complex |
| 30. | Stenotrophomonas maltophilia |
| 31. | Achromobacter xlyosoxidans |
| 32. | Internal Positive Control oligonucleotide |
| 33. | Small RNA-base Viability Assay |

High Efficiency Filtration System and Method Thereof

As discussed herein, in one embodiment, the present disclosure is directed to the portable system 10 including the portable fluid filtration device 200 for use with device 300 for rapid analysis of one or more biological samples.

Now referring to FIG. 1, the liquid source 100 comprises any number of different types of sources of fluid that is to be analyzed using the system 10 for detection of pathogens. The source 100 can take any number of different sizes and can be located at a variety of different locations. In general, the source 100 can comprise any source of contaminated water. As previously discussed contaminated water can be found in a wide array of locations including a source of drinking water, bathing water, recreation water (e.g., swimming pools and spas), air conditioning cooling towers, hot water heaters, respiratory therapy equipment, etc. In addition, natural water sources, such as springs, ponds, lakes, irrigation water, etc. can also become a source of contaminated water.

FIG. 1 illustrates that a first conduit 12 is used to deliver the liquid (water) from the source 100 to the portable filtration device 200. In some settings, the first conduit 12 can be directly inserted into the source 100, such as a water reservoir, cooling tower, pond, etc., or a sample can be taken and placed into a collection container and in that case, the first conduit 12 can be inserted into the collection container that holds the liquid sample. The first conduit 12 can be in the form of a flexible tube or the like. It will also be understood that the first conduit 12 can actually be formed of two or more tube segments that are coupled to one another with a connector or the like.

To deliver the fluid (liquid) from the source 100 to the portable filtration device 200, a pump 210 can be used and is generally disposed along the first conduit 12 to controllably pump the fluid from the source 100 to the portable filtration device 200.

Any number of different types of pumps 210 can be used including automated pumps and manual pumps, such as hand pumps, etc.

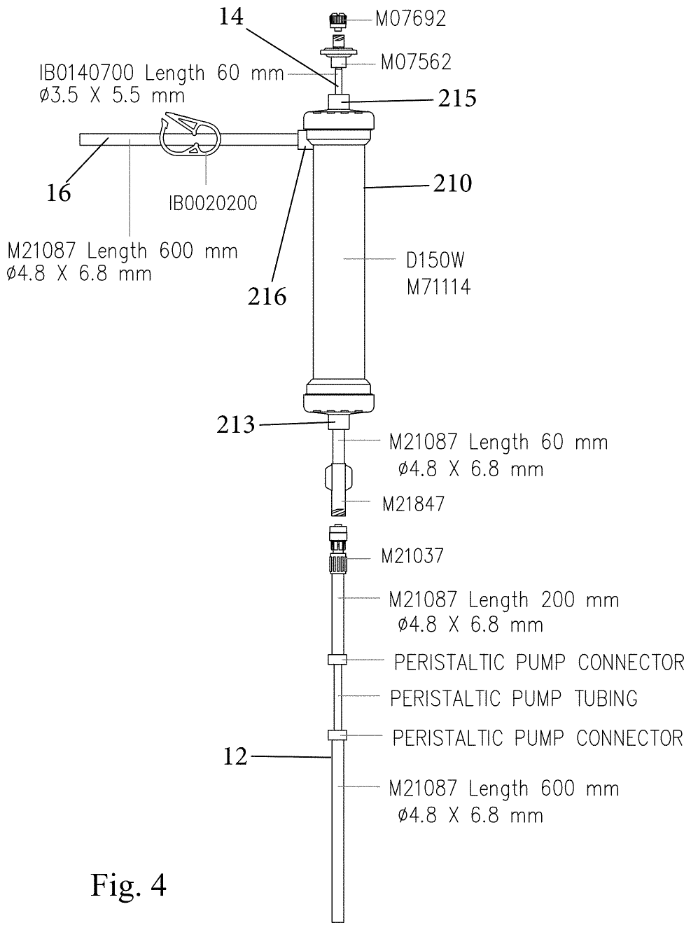

One exemplary type of pump 210 is a peristaltic pump. As is known, a peristaltic pump is a type of positive displacement pump used for pumping a fluid and also can be commonly referred to as a roller pump. The fluid is contained within a flexible tube fitted inside a circular pump casing. A rotor with a number of rollers attached to the external circumference of rotor compresses the flexible tube. As the rotor turns, the part of the tube under compression is pinched closed (occludes) thus forcing the fluid to be pumped to move through the tube. As the tube opens back to its natural state after the passing of the cam, fluid flow is induced to the pump. When a peristaltic pump 210 is used, the first conduit 12 can include a peristaltic pump tubing segment (FIG. 4) that is contacted by the rotor. The peristaltic pump 210 can be provided with a small footprint and can include a housing with an inlet and outlet connector for connecting to first conduit segments and can be powered by an electric power source or a battery power source. The pump 210 has conventional controls, such as on/off, speed, etc.

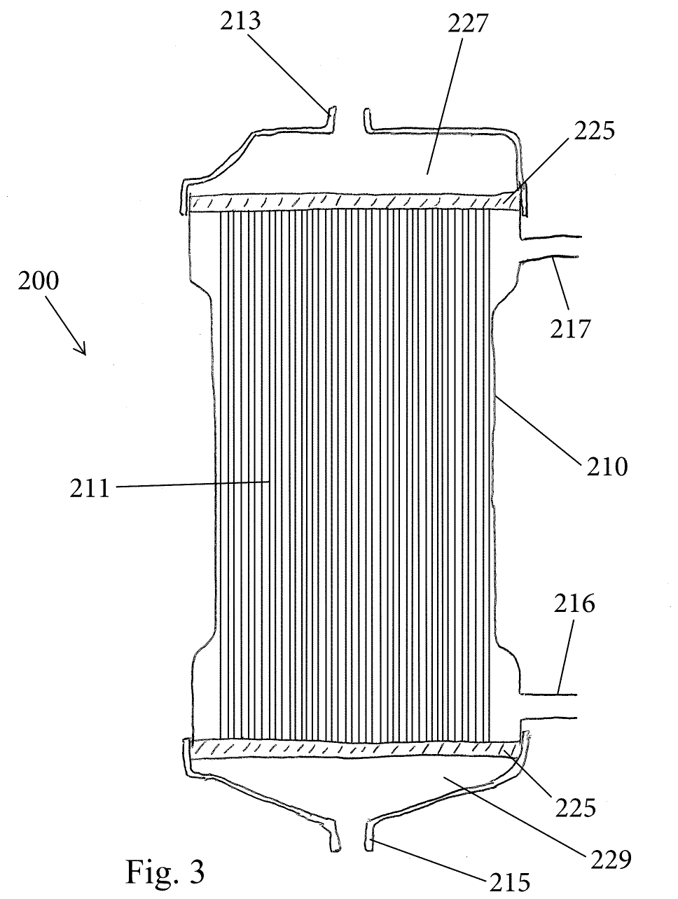

As shown in FIGS. 2 and 3, the portable filtration device 200 can come in any number of different forms that are suitable for the intended application. For example, the filtration device 200 is in the form of a cartridge that is defined by a housing 210. Housing 210 is preferably cylindrical in shape and is formed of a rigid plastic material. Housing 210 contains a longitudinal bundle of semi-permeable hollow fibers 211, as are known in the art. The semi-permeable hollow fibers 211 are configured to filter fluid by forcibly conducting fluid across the hollow fibers 211. Any number of semi-permeable hollow fibers 211 that are commercially available for this intended purpose may be used. For example, semi-permeable hollow fibers 211 come in variety of dimensions and can be formed of polymers, such as polysulfone, or be cellulose-based.

The housing 210 includes a number of different integral ports that permit fluid to enter and exit the housing 210. The housing 210 includes a first end 212 and a second end 214. At the first end 212 there is a first port 213 that can be in the form of an inlet and at the second end 214, there is a second port 215 that can be in the form of an outlet. The housing 210 further includes a third port 216 that can be located along the side of the housing 210 (FIG. 2) and optionally and according to some housing constructions, the housing 210 can include a fourth port 217 (FIG. 2). The third and fourth ports 216, 217 are in fluid communication with the hollow interior of the housing 210 that is located external to and about the hollow fibers 211. In contrast, the first and second ports 213, 215 are in fluid communication with opposing ends of the fibers 211 and more specifically are in fluid communication with the lumens thereof. Header spaces 227, 229 can be formed at the ends of the cartridge and as is known in the art, a potting compound 225 can be used to seal around the fibers 211 at the ends of the housing, while leaving the lumens of the fibers 211 in fluid communication with the open header spacers. The first port 213 is thus in fluid communication with the first header space 227 and the second port 215 is in fluid communication with the second header space 229.

In the illustrated embodiment, the first port 213 represents the inlet port for injecting fluid samples and reagents into the lumens of the fibers 211 for filtering. Thus, the first conduit 12 (which can include the peristaltic pump section when a peristaltic pump is used) is connected at one to the first port 213. Pump 210 is shown in FIG. 2 as well.

In certain embodiments, commercially available filtration device may be used to filter the fluid sample. Examples of commercially available filtration device include High Performance Antipyrogenic Ultrafilter for Replacement Solutions (D150/U) from Medica Group. (See M27053 rev.02 modifica ME190314C del 19.03.2014)

In certain embodiments, the filtration device 200 has a fiber membrane (fibers 211) with pore size from about 0.002 micron to about 0.01 micron.

In certain embodiments, the filtration device 200 has a filtration capacity to reduce the volume of an initial fluid sample by a factor of at least 10E-5.

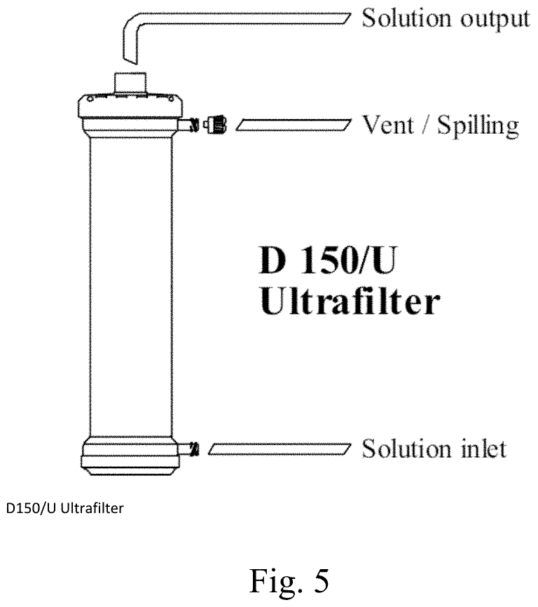

The fourth port 217 can be closed with a cap (not shown), while the third port 216 can be connected to a third conduit 16 that receives purified fluid (i.e., fluid that has been filtered across the fibers 211) and delivers the purified fluid to either a collection vessel 301, to a drain, or even can be open at its distal ends to deliver the purified fluid back to ground soil if the system 10 is being operated outside. Alternatively, the third port 216 can be capped and the third conduit 16 is connected to the fourth port 217. In the embodiment shown in FIG. 4, the cartridge only includes a third port 216 and thus, the third conduit 16 is connected to the third port 216. FIG. 5 illustrates another filtration device that can be used. In this case, the solution to be filtered is delivered into a side port that is in communication with the space around the fibers and the solution is outputted at the end of the cartridge by being conducted across the fibers into the lumens and then flowing to the open header space at the end, and venting is along a side port as shown. The operation of this cartridge is similar to the others described herein in that the target pathogen is collected as filtrate within the lumens of the fibers.

The third port 216 or the fourth port 217 where present can thus be considered to be a fluid outlet port.

The second port 215 acts as a venting port (air purge) and is connected to a second conduit 14 which can have a clamp 219 along its length for selectively closing off the second conduit 14 and also has an air valve (air check) or the like to permit air to be vented from inside the cartridge and more particularly from within the lumens. When the clamp is open, venting is permitted. It will be appreciated that the fibers 211 are initially filled with air and need to be wetted (primed) with fluid. As fluid (liquid) is delivered into the first port 213 into the lumens of the fibers 211, the air within the lumens is expelled downward to the second port 215 where it is vented. The venting mechanism in the second conduit 14 is designed so that no liquid is expelled through the second port 215 into the second conduit 14. From the second conduit 14, air is vented to atmosphere.

In operation, the system 10 is first operated to generate filtrate within the lumens of the fibers 211 by delivering liquid from the source 100 into the lumens of the fibers 211 using pump 210 or another mechanism. After the air within the lumens is purged through the second port 215. The liquid (e.g., water) is conducted across the fibers 211 and purified water exits through the third port 216 (or fourth port 217). Filtrate which comprises any target pathogens is left behind within the lumens of the fibers 211. Once a sufficient amount of liquid is filtered through the filtration device 200, the delivery of the liquid is stopped. In one embodiment, about 1 liter of liquid is filtered through the filtration device 200; however, this is merely exemplary and other volumes of liquid can be passes through the filtration device 200. For example, in one exemplary embodiment, between about 100 ml to about 100 gallons of the liquid sample can be passed through the filtration device 200. The precise amount depends on the given application (liquid source, etc.), expected concentration and also type of pathogens to be concentrated and collected as filtrate, etc.

For example, the first conduit 12 can be removed from the source 100.

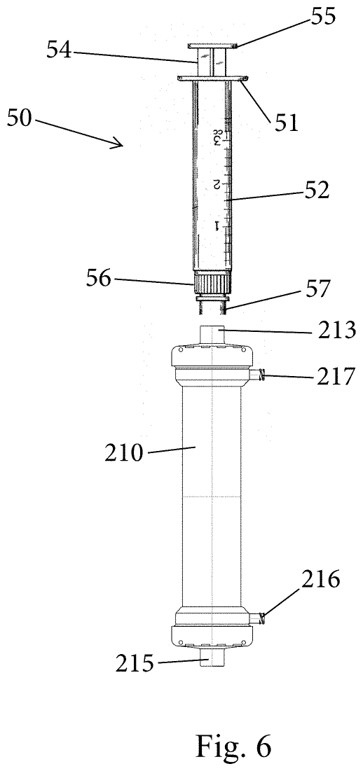

Once this step is performed, the filtrate (in residue form) is then processed and collected by treating the filtrate to lyse the target pathogen nucleus therein and collect the lysed target pathogen (FIG. 6). This step can be performed by disconnecting the first conduit 12 from the first port 213 and then connecting a device for delivering a liquid into the lumens of the fibers to contact the filtrate. For example, and as shown in FIG. 6, a syringe 50 can be used (for simplicity, the conduits are not shown connected to the cartridge in FIG. 6). Syringe 50 can be a conventional syringe that includes a barrel 52 and a slidable plunger 54 that is inserted into the barrel 52. A flange 51 extends from the barrel 52 for holding the barrel 52 and a flange 55 extends from the plunger 54. At a distal end of the barrel 52, a first connector 56 is present and can mate with a connector part 57, such as a Luer lock, that is configured to connect to the first port 213 in a sealed manner.

Initially, the barrel 52 contains the liquid that is used to lyse the target pathogen(s). In one embodiment, about 20 ml of buffer solution can be used and is entirely injected into the lumens of the hollow fibers. The lumens of the hollow fibers of one exemplary cartridge contain about 15 ml of space and therefore, if 20 ml of buffer is used, some of the buffer gets pushed through the filter (hollow fibers). The barrel 52 is fluidly connected to the first port 213 and then the plunger 54 is manipulated to deliver (expel) the liquid into the lumens of the fibers 211 for contacting the target pathogens (filtrate/residue). The plunger 54 preferably is slowly moved to ensure a slow delivery of the liquid to allow for proper lysing of the pathogens.

In certain embodiments, this lysis treatment involves a lysis buffer comprising: 4.5 M GITC (guanidinium isothiocyanate) dissolved in Tris(10 mM)-EDTA (1 mM) (TE) buffer (pH 8.0) polyadenylic acid [poly(A)] (17.6 μg/mL); 0.14 M sodium acetate (NaOAc); 0.24 M NaCl; 0.4% sodium sulphite; 0.2% dithioerythritol (DTE); 0.02% Sodium dodecyl sulfate (SDS); and 0.4% Tween 20.

In certain embodiments, lysis buffer is slowly inserted to the filtration device 200 manually via the syringe 50 to absorb the filtrate, over the time period of about 5 to 60 seconds. As mentioned, in one embodiment, about 20 ml of buffer is delivered (broadly speaking the volume of buffer delivered is greater than the inner volume of the lumens of the hollow fibers and can be slightly greater such as 10% or 25% or 50% greater).

Once the lysis treatment is completed, the user then moves the plunger 54 in the opposite direction so as to pull the solution within the lumens of the fibers 211 back into the barrel 52 of the syringe 50 so as to collect the lysed target pathogen solution within the barrel 52.

When the volume (inner space) of the lumens of the hollow fibers is about 15 ml, the extraction of the solution results in about 15 ml or more of the solution being extracted.

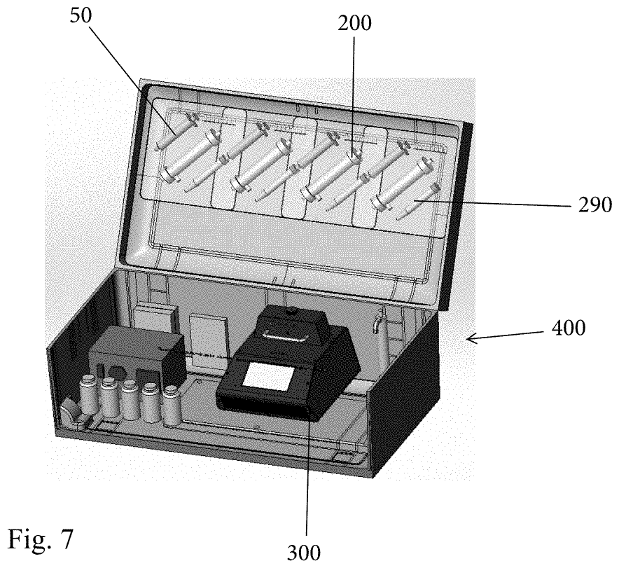

Next, the lysed target pathogen solution can be placed into a container or vessel by pushing the plunger 54 forward to expel the lysed target pathogen solution into the container. The container can be of a type that mates with the connector 57. The container can be in the form of a reservoir tube 290 shown in FIG. 7.

Next, a prescribed amount of lysed target pathogen solution is removed from the container (reservoir tube) and delivered into a well or the like that is part of the molecular detection device 300. For example, an amount of between about 10 microliters and 50 microliters can be aliquoted from the container and then delivered into the one or more test wells of the molecular detection device 300. The wells of the molecular detection device 300 contain lyophilized primers, cap oligos, probes and master mix for polymerase chain reaction detection and are reconstituted with the lysed pathogen solution. The wells are thus selectively formed in view of the target pathogens that are being tested in the liquid sample. It will therefore be appreciated that in kit form, a variety of different target pathogen tests and combinations thereof can be configured in specific wells and combinations of wells. The wells are individually identified with indicia so that the user knows which wells are being used.

The molecular detection device 300 incubates the lysed target pathogen solution under amplification conditions with oligonucleotide primers and DNA polymerase; and is configured to detect amplified target DNA to determine the presence or absence as well as the quantification in the fluid sample of the target pathogens or indicator microorganisms carrying the selected target DNA nucleotide sequence.

Any number of suitable molecular detection devices 300 can be used and preferably, as discussed herein, the molecular detection device 300 preferably has a small footprint and is portable. In some embodiments, the device 300 can be battery powered.

Molecular Detection Device—PCR

Amplification of the target pathogen's DNA sequence is by means of selected primer pairs according to a procedure known as Polymerase Chain Reaction, hereinafter referred to simply as PCR. PCR amplification of nucleotide sequences is described in U.S. Pat. No. 4,683,202, the disclosure of which is incorporated herein by reference. The PCR amplification process comprises amplifying a selected or targeted nucleic acid sequence by treating the two separate complementary strands of the nucleic acid sequence with two oligonucleotide primers, each being complementary to one of the two strands, to anneal the primers to their complementary strands, then synthesizing extension products of said primers by polymerase to extend said primers to make fully double-stranded replicas of the selected target nucleic acid sequence, followed by separation (denaturation) of the extension products and repeating this amplification sequence the desired number of cycles to increase the concentration of the selected nucleic acid sequence. The process is utilized for detection of DNA fragments in a sample.

In one embodiment, described herein is a method of for analysis of a fluid sample to detect the presence of target pathogens or indicator microorganisms using a molecular detection device, comprising steps of: preparing test wells containing lyophilized primers, cap oligos, probes and master mix for polymerase chain reaction detection; preparing reverse transcribed-RNA-based test wells for target pathogen specific detection (RNA viruses) or in the detection of bacterial cell viability, containing lyophilized primers, cap oligos, probes, transcription factors and master mix; placing test wells into a multi-well, multi-channel thermocycling device and running them on repeated heat cycle pattern; calculating the concentration of colony or plaque forming units in each test sample, using quantification of nucleic acid in fluorescence units for each specific target pathogen.

In certain embodiment, the first heating cycle is run for between 30 and 180 seconds longer than the standard heating cycles to further break-up potential cellular walls and free DNA fragments.

In certain embodiment, subsequent cycles are standard heating cycles for up to 40 cycles to facilitate the polymerase chain reactions to detect the nucleic acid fragments of interest.

Probes

In addition to the probe nucleotide sequence, the probe can comprise additional nucleotide sequences or other moieties that do not inhibit the disclosed methods. In convenient embodiments, the probe can comprise additional nucleotide sequences or other moieties that facilitate the disclosed methods. For instance, the probe can be blocked at its 3′ terminus to prevent undesired nucleic acid polymerization priming by the probe. Also, moieties may be present within the probe that stabilize or destabilize hybridization of the probe or probe fragments with the nucleotide sequence. The probes can also comprise modified, non-standard, or derivatized nucleotides.

In certain embodiments, the probe can comprise a detectable moiety. The detectable moiety can be any detectable moiety known by one of skill in the art without limitation. Further, the detectable moiety can be detectable by any means known to one of skill in the art without limitation. For example, the detectable moiety can be detectable by spectroscopic, photochemical, biochemical, immunochemical, or chemical means.

A variety of detectable moieties that can be used to detect the probes, as well as methods for their linkage to the probe, are known to the art and include, but are not limited to, enzymes (e.g., alkaline phosphatase and horseradish peroxidase) and enzyme substrates, radioactive moieties, fluorescent moieties, chromophores, chemiluminescent labels, electrochemiluminescent labels, such as Origin™ (Igen, Rockville, Md.), ligands having specific binding partners, or any other labels that may interact with each other to enhance, alter, or diminish a signal. Should a 5′ nuclease reaction be performed using a thermostable DNA polymerase at elevated temperatures, the detectable moiety should not be degraded or otherwise rendered undetectable by such elevated temperatures.

In certain embodiments, the detectable moiety can be a fluorescent moiety. The fluorescent moiety can be any fluorescent moiety known to one of skill in the art without limitation. In general, fluorescent moieties with wide Stokes shifts are preferred, allowing the use of fluorometers with filters rather than monochromometers and increasing the efficiency of detection. In certain embodiments, the fluorescent moiety can be selected from the group consisting of fluorescein-family dyes (Integrated DNA Technologies, Inc., Coralville, Iowa), polyhalofluorescein-family dyes, hexachlorofluorescein-family dyes, coumarin-family dyes (Molecular Probes, Inc., Eugene, Or), rhodamine-family dyes (Integrated DNA Technologies, Inc.), cyanine-family dyes, oxazine-family dyes, thiazine-family dyes, squaraine-family dyes, chelated lanthanide-family dyes, BODIPY®-family dyes (Molecular Probes, Inc.), and 6-carboxyfluorescein (FAM™) (Integrated DNA Technologies, Inc.). Other examples of fluorescent moieties that can be used in the probes, methods, and kits can be found in U.S. Pat. Nos. 6,406,297, 6,221,604, 5,994,063, 5,808,044, 5,880,287, 5,556,959, and 5,135,717.

In other embodiments, the detectable moiety can be a detectable moiety other than a fluorescent moiety. Among radioactive moieties, ³²P-labeled compounds are preferred. Any method known to one of skill in the art without limitation may be used to introduce ³²P into a probe. For example, a probe may be labeled with ³²P by 5′ labeling with a kinase or by random insertion by nick translation. Detectable moieties that are enzymes can typically be detected by their activity. For example, alkaline phosphatase can be detected by measuring fluorescence produced by action of the enzyme on appropriate substrate compounds. Where a member of specific binding partners are used as detectable moieties, the presence of the probe can be detected by detecting the specific binding of a molecule to the member of the specific binding partner. For example, an antigen can be linked to the probe, and a monoclonal antibody specific for that antigen can be used to detect the presence of the antigen and therefore the probe. Other specific binding partners that can be used as detectable moieties include biotin and avidin or streptavidin, IgG and protein A, and numerous other receptor-ligand couples well-known to the art. Still other examples of detectable moieties that are not fluorescent moieties can be found in U.S. Pat. Nos. 5,525,465, 5,464,746, 5,424,414, and 4,948,882.

The method of linking or conjugating the detectable moiety to the probe depends, of course, on the type of detectable moiety or moieties used and the position of the detectable moiety on the probe.

The detectable moiety may be attached to the probe directly or indirectly by a variety of techniques. Depending on the precise type of detectable moiety used, the detectable moiety can be located at the 5′ or 3′ end of the probe, located internally in the probe's nucleotide sequence, or attached to spacer arms of various sizes and compositions to facilitate signal interactions. Using commercially available phosphoramidite reagents, one can produce oligonucleotides containing functional groups (e.g., thiols or primary amines) at either terminus via an appropriately protected phosphoramidite, and can attach a detectable moiety thereto using protocols described in, for example, PCR Protocols: A Guide to Methods and Applications, ed. by Innis et al., Academic Press, Inc., 1990.

In certain embodiments, the detectable moiety can be attached to the 5′ end of the probe. In certain embodiments, the detectable moiety can be attached to the 3′ end of the probe. In other embodiments, the detectable moiety can be attached to the probe at a residue that is within the probe. The detectable moiety can be attached to any portion of a residue of the probe. For example, the detectable moiety can be attached to a sugar, phosphate, or base moiety of a nucleotide in the probe. In other embodiments, the detectable moiety can be attached between two residues of the probe.

In certain embodiments, the probe can comprise a fluorescent moiety and a quencher moiety. In such embodiments, the fluorescent moiety can be any fluorescent moiety known to one of skill in the art, as described above. Further, the quencher moiety can be any quencher moiety known to one of skill in the art without limitation. In certain embodiments, the quencher moiety can be selected from the group consisting of fluorescein-family dyes, polyhalofluorescein-family dyes, hexachlorofluorescein-family dyes, coumarin-family dyes, rhodamine-family dyes, cyanine-family dyes, oxazine-family dyes, thiazine-family dyes, squaraine-family dyes, chelated lanthanide-family dyes, BODIPY®-family dyes, and non-fluorescent quencher moieties. In certain embodiments, the non-fluorescent quencher moieties can be BHQT™-family dyes (including the quenchers described in WO 01/86001), Iowa Black™. or Dabcyl (Integrated DNA Technologies, Inc.). Other examples of specific quencher moieties include, for example, but not by way of limitation, TAMRA (N,N,N′,N′-tetramethyl-6-carboxyrhodamine) (Molecular Probes, Inc.), DABCYL (4-(4′-dimethylaminophenylazo)benzoic acid), Iowa Black™. (Integrated DNA Technologies, Inc.), Cy3™ (Integrated DNA Technologies, Inc.) or Cy5™ (Integrated DNA Technologies, Inc.). Other examples of quencher moieties that can be used in the probes, methods, and kits can be found in U.S. Pat. Nos. 6,399,392, 6,348,596, 6,080,068, and 5,707,813.

In certain embodiments, the quencher moiety can be attached to the 5′ end of the probe. In certain embodiments, the quencher moiety can be attached to the 3′ end of the probe. In other embodiments, the quencher moiety can be attached to the probe at a residue that is within the probe. The quencher moiety can be attached to any portion of a residue of the probe. For example, the quencher moiety can be attached to a sugar, phosphate, or base moiety of a nucleotide in the probe. In other embodiments, the quencher moiety can be attached between two residues of the probe.

While probes can be used for the quantification of pathogens in qPCR, other methods can used for quantification including but not limited to the use of SYBR® Green or Chai Green®.

Quantification of Target Pathogen

Described herein is a method of quantifying nucleic acid in fluorescence units for each specific target pathogen to calculate the concentration of colony or plaque forming units in each test sample.

The concentration of target pathogen's nucleic acid molecules is determined by the number of nucleic acid molecules of the assay target pathogen present in the polymerase chain reaction volume. The number of nucleic acid molecules present is equal to

N₀=Nₜ/(E+1)^{Ct}

where Cₜ is the fractional threshold cycle as determined by the fluorescent signal above baseline, Nₜ is the number of amplicon molecules at fluorescent threshold, and E is amplification efficiency, compared to replicate standard curves for the assay. The concentration of nucleic acid is equal to

Aₙₐ=N₀/Vₚ
where Aₙₐ is the concentration of nucleic acid and Vₚ is the polymerase chain reaction volume.

The corresponding concentration of colony or plaque forming units is determined by the number of copies of the assay target nucleic acid normally present in the viable target pathogen and is equal to A_{fu}=Aₙₐ/A_{ba} where A_{fu} is the concentration of colony forming units in the polymerase chain reaction volume in microliters and A_{ba} is the average or normal number of copies of the assay target nucleic acid present in the viable target pathogen.

For the A_{fu} value to be practically useful and meaningful to scientific, regulatory, and public health standards for the presence of target pathogens in a fluid sample (i.e., water), it remains to calculate the concentration of estimated target pathogen colony or plaque forming units per milliliter for comparison. The concentration of target pathogen colony or plaque forming units per milliliter in the original fluid sample taken is equal to:

Aₛₐₘₚₗₑ=(A_{fu}*V_{con}*10³)/V_{orig}

where V_{con} is the volume of the concentrated lysate solution collected in milliliters and V_{orig} is the volume of the fluid sample collected in milliliters.

Fluid Analysis Kit

Described herein is a kit for use in a process for analyzing fluid sample the presence of target pathogens or indicator microorganisms, comprising; the filtration device 200, collection conduit 12, peristaltic pump device 210, dispensing conduit 16, and the molecular detection device 300.

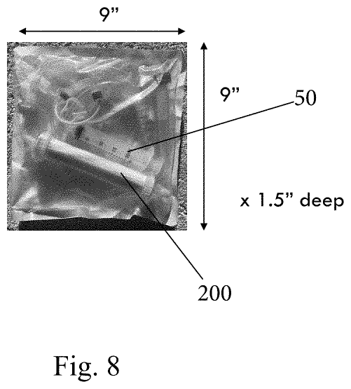

FIG. 7 shows a kit 400 that includes an openable portable case that contains the components of the system 10. The molecular detection device 300 can be provided along a floor of the case. Plural portable filtration devices 200 can be mounted along a lid of the case along with other components such as syringes 50 and containers 290. Tubing and the like can also be provided in the case along with pump 210 which can be battery driven to allow portable use outside of where electricity is available. The molecular detection device 300 can also be battery powered. FIG. 8 shows an individual vacuum sealed package that contains certain components, such as one filtration device 200 and one syringe 50 and can include one container 290. By being prepackaged, the user can simply take one package to perform one test of one sample. To perform a second test of a second sample, another package is accessed.

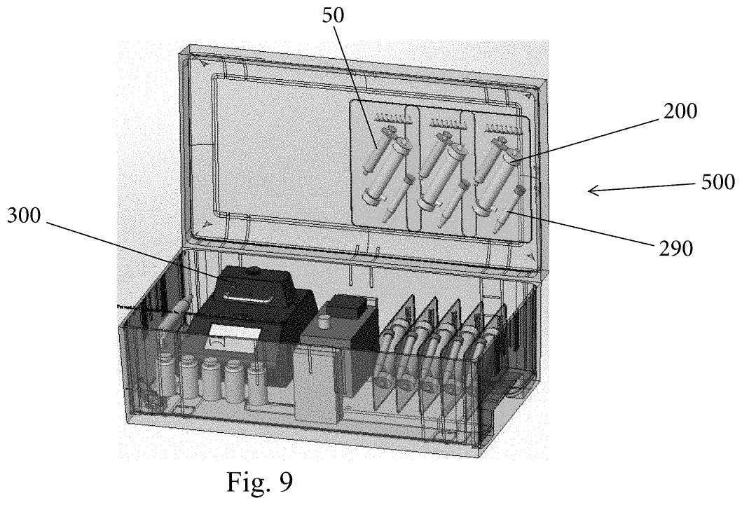

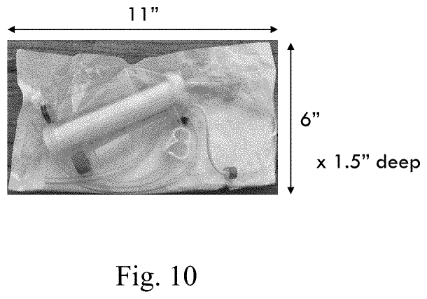

FIG. 9 shows another kit 500 similar to the kit 400 and includes components 200, 50, 290 of the system and can include all other components, such as the conduits (tubing), etc. FIG. 10 shows another individual sealed package.

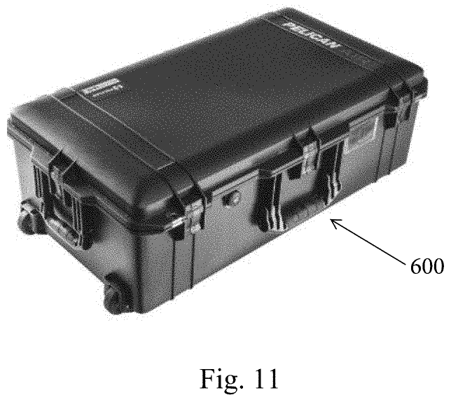

FIG. 11 shows that the system can be contained with a hard shell case 600.

In one embodiment, the kit comprises an outside carrier with a hard surface. (FIG. 11) In one embodiment, the kit is portable. In certain embodiments, commercially available outside carrier may be used. Examples of commercially available outside carrier include 1615 Air Case from Pelican. (See https://www.pelican.com/us/en/product/cases/air/1615)

In one embodiment, the kit includes a hardware mobile electronic device and a software application that provide a graphical user interface for human interaction, comprising information inputs and reporting outputs.

Mobile Analysis Device and Application

Described herein is a hardware mobile electronic device and a software application that provide a graphical user interface for human interaction, comprising information inputs and reporting outputs. In one embodiment, the mobile electronic device and the application communicate wirelessly with the molecular detection device 300 as well as a web-based data storage and analytic computing capability. In one embodiment, the mobile electronic device 300 and the application wirelessly transmit the programming of the polymerase chain reaction thermocycling device (device 300), collect and associate sample identification and associated metadata, wirelessly retrieve the results of the polymerase chain reaction analysis from the thermocycling device, transmit the results to a web-based computer platform to perform the calculations to determine the colony or plaque forming units per milliliter in the original fluid sample and generation of result reports for printing or electronic dissemination.

In this regard, the molecular detection device 300 can include a microprocessor that is configured to control the various components of the device 300 and carry out aspects of the systems and methods disclosed herein. The microprocessor can be a number of processors, a multi-processor core, or some other type of processor, depending on the particular implementation. In some implementations the microprocessor is configured by executing one or more software modules that can be loaded into a memory and executed by the microprocessor. The one or more software modules can comprise one or more software programs or applications having computer program code or a set of instructions executed in the microprocessor. Such computer program code or instructions can be written in any combination of one or more programming languages. Preferably, included among the software modules are a user input module, a display module, a stimuli control module and a communication module. During execution of the software modules, the microprocessor configures the device 300 to perform various operations described herein.

Memory can be, for example, a random access memory (RAM) or any other suitable volatile or non-volatile computer readable storage medium. In addition, memory can be fixed or removable and can contain one or more components or devices such as a hard drive, a flash memory, a rewritable optical disk, a rewritable magnetic tape, or some combination of the above. In addition, memory can be onboard the microprocessor. In addition, it should be noted that other information and/or data relevant to the operation of the present systems and methods can also be stored on memory, as will be discussed in greater detail below.

A display (e.g., LCD display) can also be operatively connected to the microprocessor. The display can be a digital display such as a segment display, a dot matrix display or a 2-dimensional display and can incorporate, by way of example and not limitation, a liquid crystal display, light emitting diode display, electroluminescent display and the like. The display provides an output to the user of information relevant to the operation of the device 300.

A control button and touch interface represent one or more user input devices that are operatively connected to the microprocessor. Such user input devices serve to facilitate the capture commands from the user such as an on-off commands and operating parameters related to the operation of the device. User input devices can also serve to facilitate the capture of other information from the user and provide the information to the microprocessor.

The control button can be one or more switch(es), button(s), knob(s), key(s). The touch interface is a touch sensitive device that can be is placed in register on the top of the display or on/around the perimeter of the display or anywhere on the housing. A touch interface is comprised of one or more thin, transparent layers that can detect when and where a user touches the interface and it allows a user to interact directly with what is displayed without requiring an intermediate device such as a computer mouse. The touch interface can be constructed using, by way of example and not limited to, resistive, capacitive, acoustic, infrared, optical imaging, or dispersive signal technology.

By way of further example, the touch interface and display can be integrated into a touch screen display. Accordingly, the screen is used to show a graphical user interface, which can display various fields or virtual buttons that allow for the entry of information by the user. Touching the touch screen at locations corresponding to the display of a graphical user interface allows the person to interact with the device to enter data, change settings, control functions, etc. So, when the touch screen is touched, interface communicates this change to microprocessor, and settings can be changed or user entered information can be captured and stored in the memory.

A communication interface can also be operatively connected to the microprocessor. The communication interface can be any interface that enables communication between the device 300 and external devices, machines and/or elements including a user's computer system. Communication interface can include but is not limited to a Bluetooth, or cellular transceiver, a radio transceiver, an NFC transceiver, a satellite communication transmitter/receiver, an optical port and/or any other such interfaces for wirelessly connecting the device 300 to an external computing device, such as a tablet, laptop, etc.

It can be appreciated that aspects of the present systems and methods can take the form of an entirely hardware embodiment, an entirely software embodiment (including firmware, resident software, micro-code, etc.), or an embodiment combining software and hardware. One of skill in the art can appreciate that a software process can be transformed into an equivalent hardware structure, and a hardware structure can itself be transformed into an equivalent software process. Thus, the selection of a hardware implementation versus a software implementation is one of design choice and left to the implementer. For example, the microcontroller can take the form of a circuit system, an application specific integrated circuit (ASIC), a programmable logic device, or some other suitable type of hardware configured to perform a number of operations. With a programmable logic device, the device is configured to perform the number of operations. The device can be reconfigured at a later time or can be permanently configured to perform the number of operations. Examples of programmable logic devices include, for example, a programmable logic array, programmable array logic, a field programmable logic array, a field programmable gate array, and other suitable hardware devices. With this type of implementation, software modules can be omitted because the processes for the different embodiments are implemented in a hardware unit.

### EXAMPLES

The present disclosure may be better understood by reference to the following non-limiting example, which is presented in order to more fully illustrate the preferred embodiments of the disclosure. They should in no way be construed to limit the broad scope of the disclosure.

### Example 1—Exemplary Protocol for qPCR of E. Coli K-12

PCR amplification is performed using a Qiagen One Step RT-PCR Kit (Cat #210210). The PCR solution contains a master mix provided in Table 2.

TABLE 2
Master Mix. Volume of sample: 5-10 ul for a final
reaction (Master Mix + sample) volume of 25 ul.

| | | Volume for |
|---|---|---|
| Reagents | [Final] | 1X (ul) |
| Qiagen One Step RT-PCR buffer, 5X | 1X | 5 |
| dNTP mix (10 mM) | 400 uM | 1 |
| Target A Primer/Probe Mix | 0.5 uM/0.3 uM | 1 |
| Target B Primer/Probe Mix | 0.5 uM/0.3 uM | 1 |
| RT Enzyme | | 1 |
| dH2O | — | 6-11 |

| | |
|---|---|
| Total Volume of Master Mix | 15-20 |

A region of an E. Coli K-12 gene is amplified using primer/probe provided in Table 3 for each target.

TABLE 3
Primer/Probe

| | | |
|---|---|---|
| SEQ. ID NO. 1 | Forward | ATCAACAAAGCC |
| (E. Coli) | Primer | CAGAAGCAGAAA |
| SEQ. ID NO. 2 | Reverse | AATATTCCCATCAGT |
| (E. Coli) | Primer | ATCACTATTTTATG |
| SEQ. ID NO. 3 | Probe | CCCTTGTAGTTACTGA |
| (E. Coli) | | ATCTGACACCGAATT |

TABLE 4
Panel Fluorophores

| Target | Fluorophore | Excitation (nm) | Emission (nm) |
|---|---|---|---|
| Target 1 | FAM | 495 | 520 |
| Target 2 | HEX | 538 | 555 |

A Primer/Probe Mix is prepared according to Table 5.

TABLE 5
Primer/Probe Mix

| Reagents | Final concentration | |
|---|---|---|
| Forward Primer | 13.3 | uM |
| Reverse Primer | 13.3 | uM |
| Probe | 6.65 | uM |
| 1X TE (pH 8.0) | Variable | |
| Total Volume | 100 | ul |

15-20 μL of the master mix is placed into the appropriate number of qPCR tube(s). 5-10 μL of sample DNA/RNA (1 pg-1 ng) or RNase-free water (for no template control reactions) is added into each qPCR tube.
The tubes are inserted into a real time thermocycler with the cycling conditions provided in Table 6. In certain embodiment, the ramp rate is 3° C./second.

TABLE 6
Cycling conditions

| | | |
|---|---|---|
| Cycling Conditions | Time | Cycles |

| | | | |
|---|---|---|---|
| 50° C.* | 30 | min | |
| 95° C. | 15 | min | |
| 95° C. | 45 | sec | 40X |
| 60° C. | 45 | sec | |

### Example 2—Comparison of Multi-Pathogen PCR Test in Unfiltered and Unconcentrated Water and Filtered and Concentrated Water—Test 1

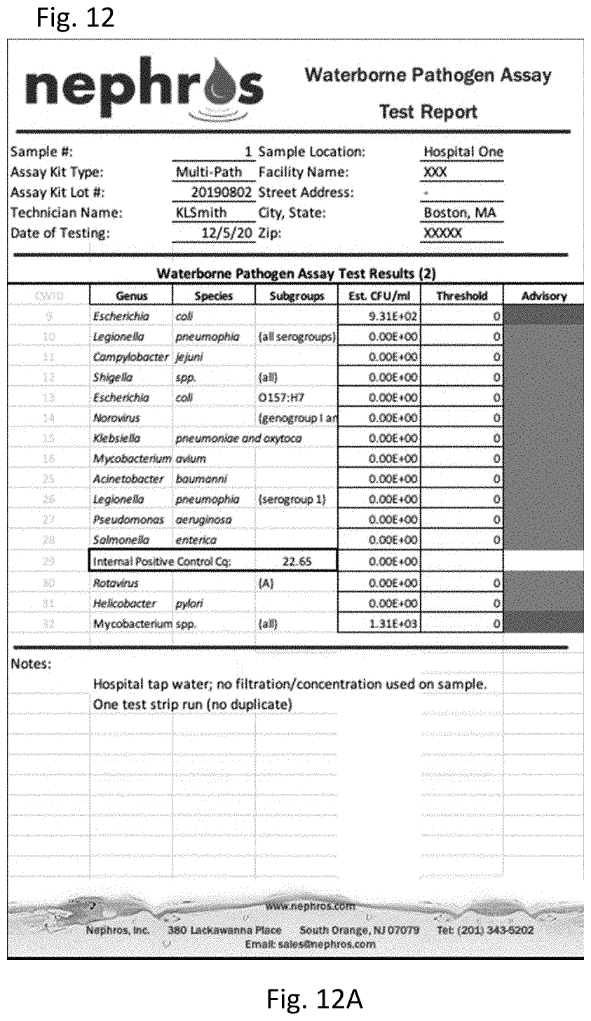

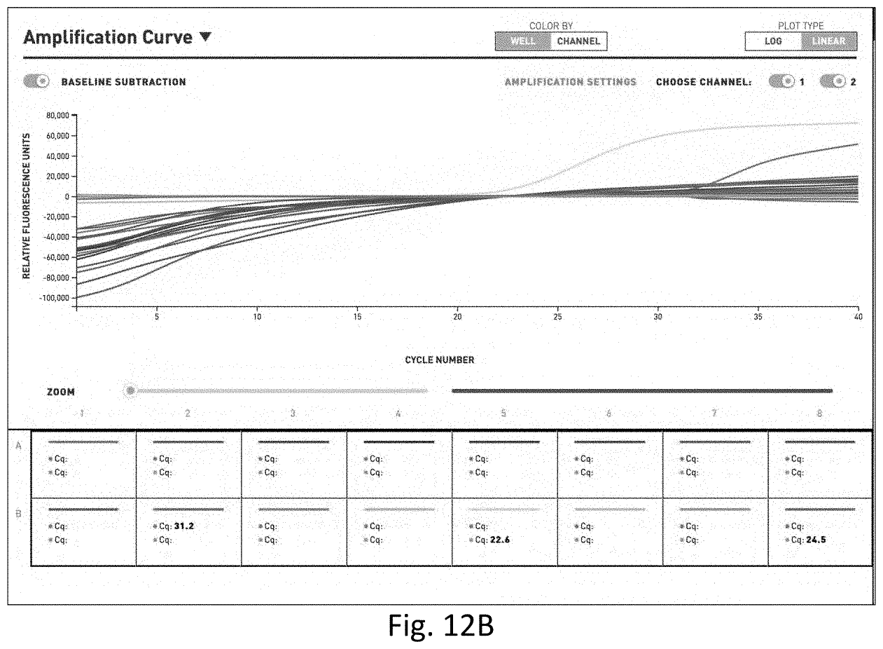

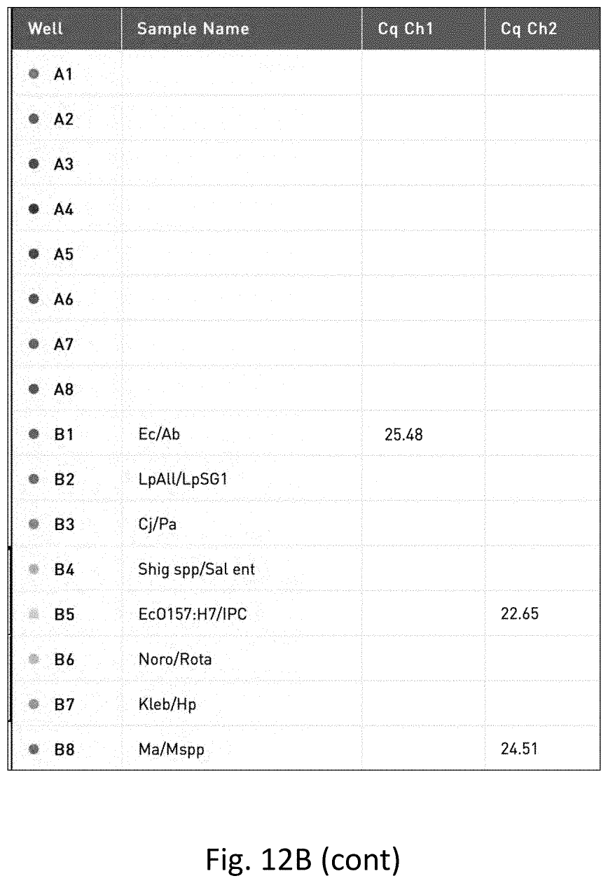

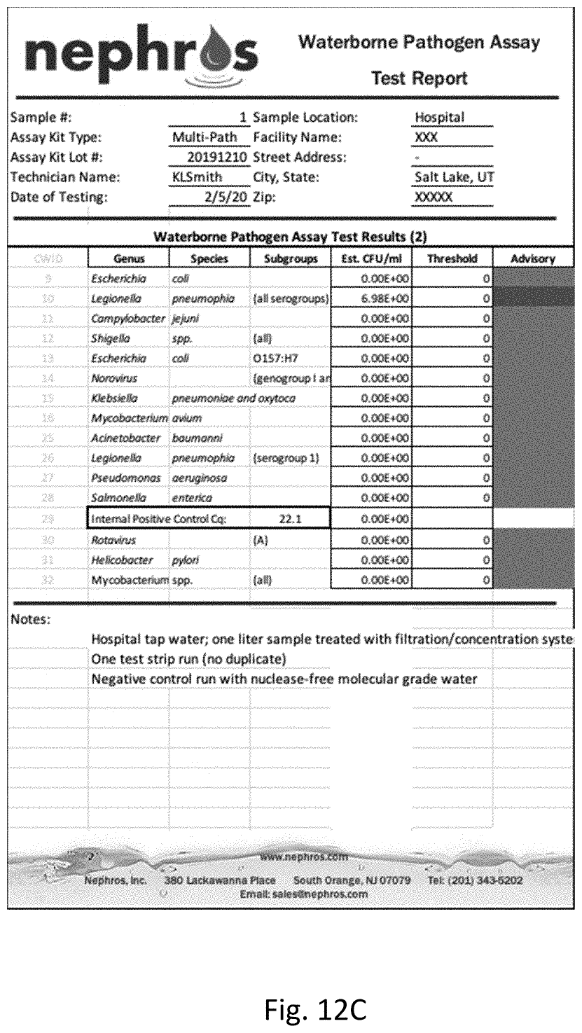

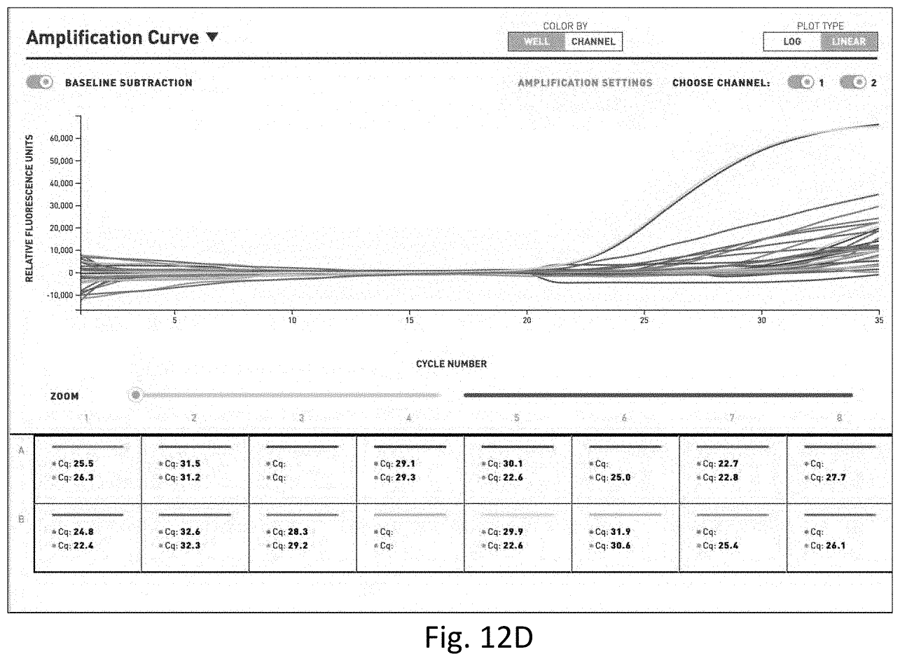

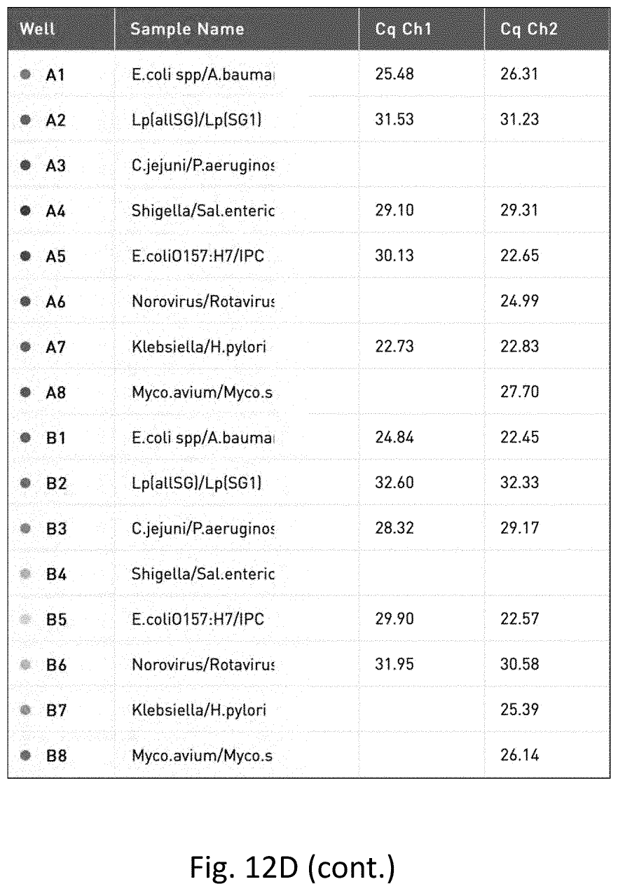

One liter of hospital tap water was tested for pathogens using a multi-pathogen PCR test using the general method of Example 1, using primers and probes for the pathogens listed in the table in FIGS. 12A and 12C. Pathogens tested for include Escherichia coli, Legionella pneumophia, Campylobacter jejuini, Shigella, Norovirus and mycobacterium.

FIGS. 12A and 12B show the results of the multi-pathogen PCR test on water that was unfiltered and unconcentrated. As shown, the only pathogens detected in this sample were E. coli and mycobacterium.

In contrast, FIGS. 12C and 12D show the results of the multi-pathogen PCR test (run in duplicate) on water that was filtered and concentrated as described herein where nine pathogens not detected in the unfiltered and unconcentrated water sample were detected.

### Example 3—Comparison of Multi-Pathogen PCR Test in Unfiltered and Unconcentrated Water and Filtered and Concentrated Water—Test 2

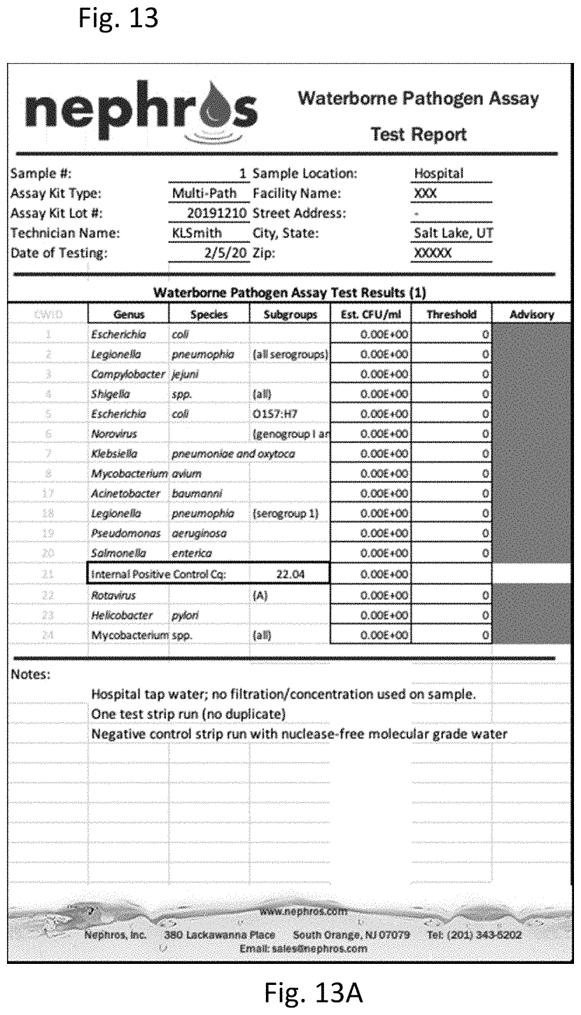

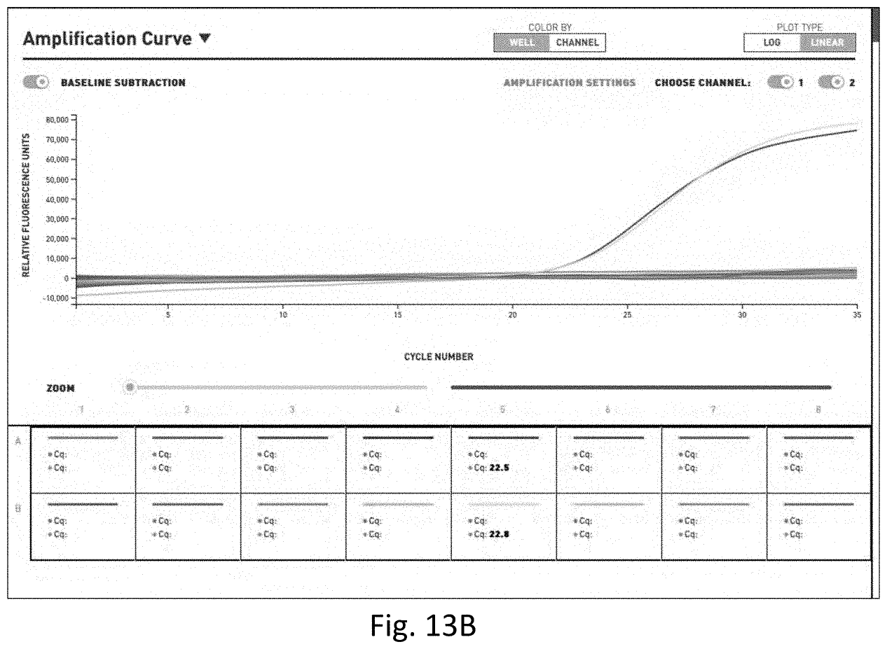

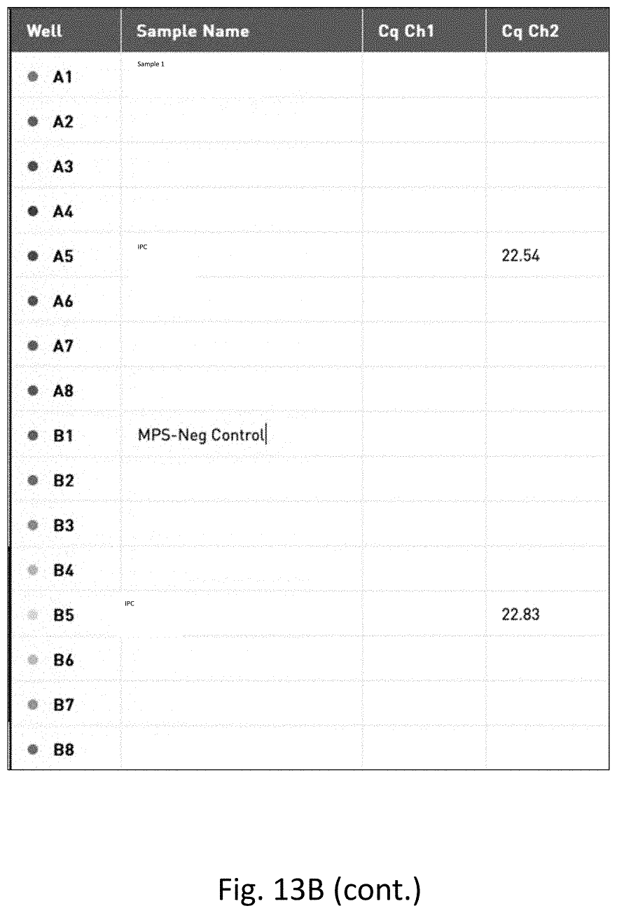

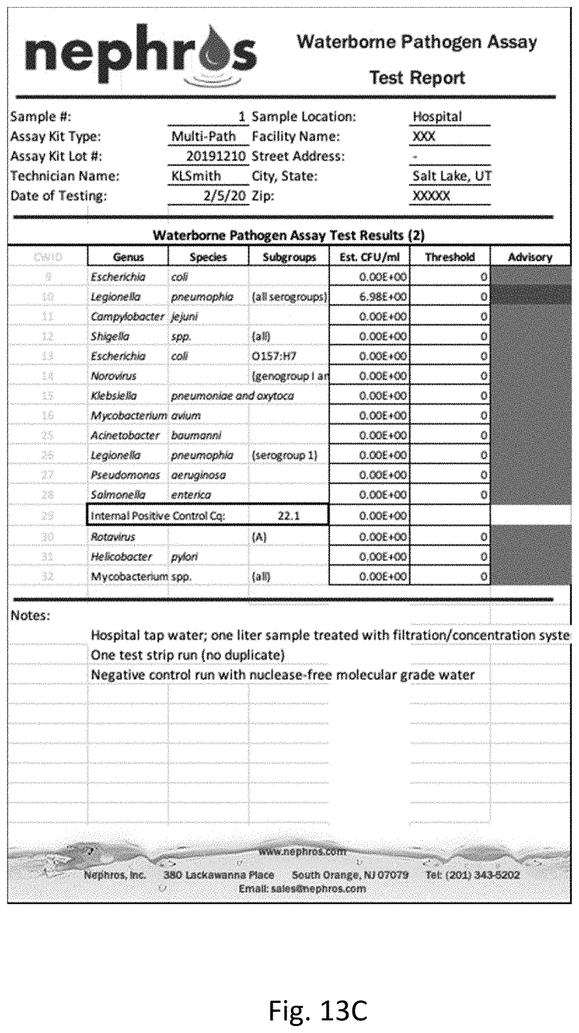

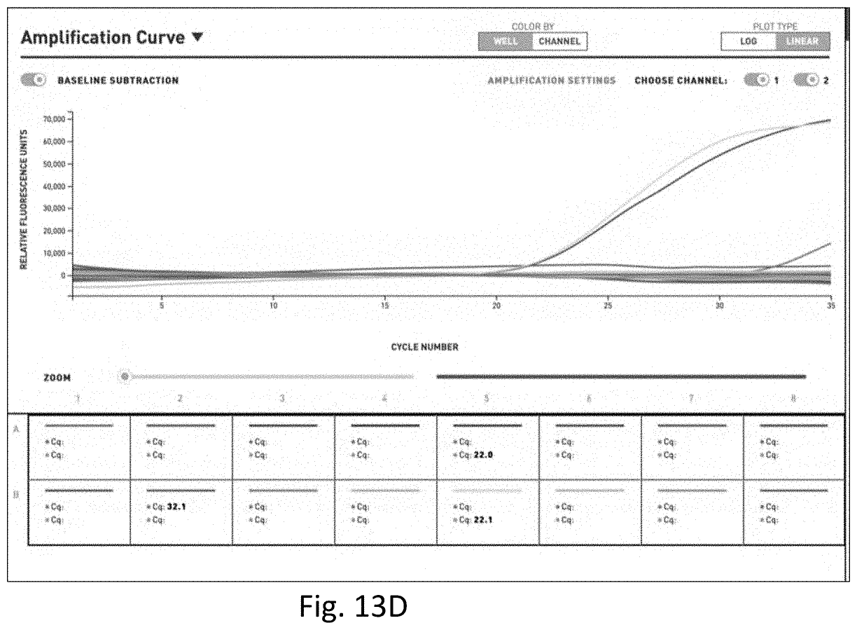

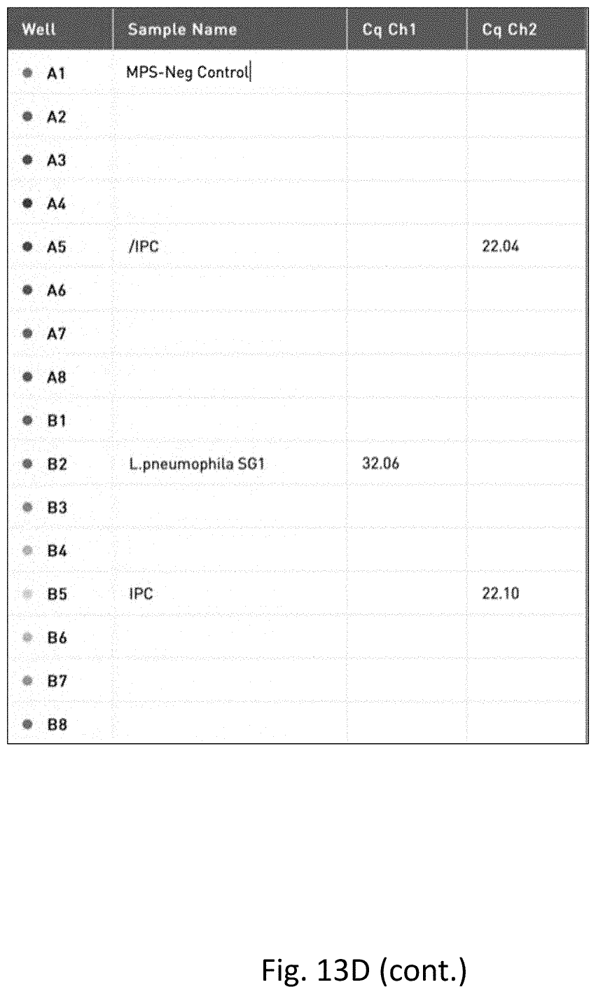

One liter of hospital tap water was tested for pathogens using a multi-pathogen PCR test using the general method of Example 1, using primers and probes for the pathogens listed in the table in FIGS. 13A and 13C. Pathogens tested for include Escherichia coli, Legionella pneumophia, Campylobacter jejuini, Shigella, Norovirus and mycobacterium. A negative control run was performed with nuclease-free molecular grade water.

FIGS. 13A and 13B show the results of the multi-pathogen PCR test on water that was unfiltered and unconcentrated. As shown, no pathogens were detected in this sample.

In contrast, FIGS. 13C and 13D show the results of the multi-pathogen PCR test on water that was filtered and concentrated as described herein where Legionella pneumophia was detected and quantitated in the filtered and concentrated sample.

### Example 4—Results of Gram Negative Pathogen Targeted PCR

Ten liter water samples were filtered and concentrated as described herein and low level dilutions were made (1 colony forming unit (CFU) per 100 ml. of E. coli). A gram-negative pathogen targeted quantitative PCR was performed using the general method of Example 1.

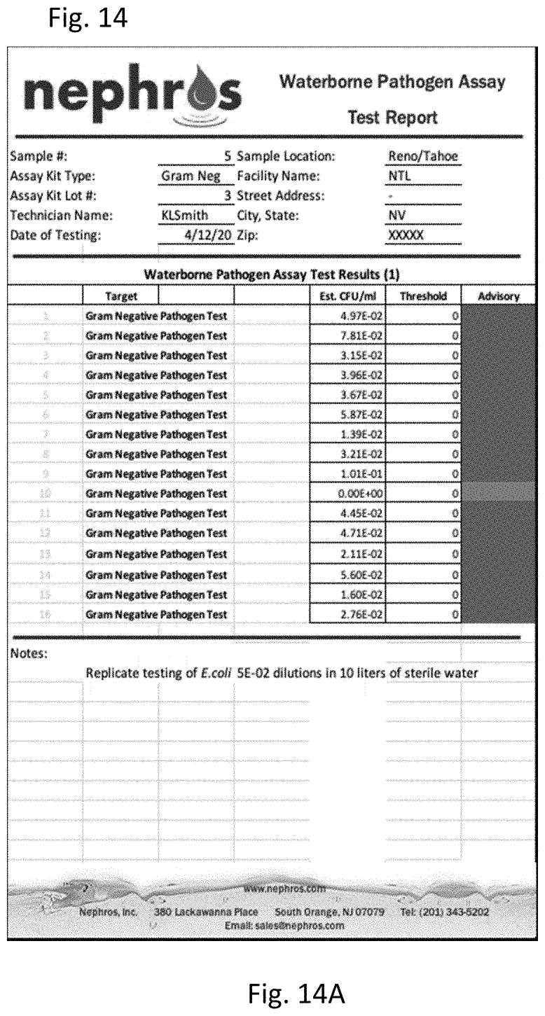

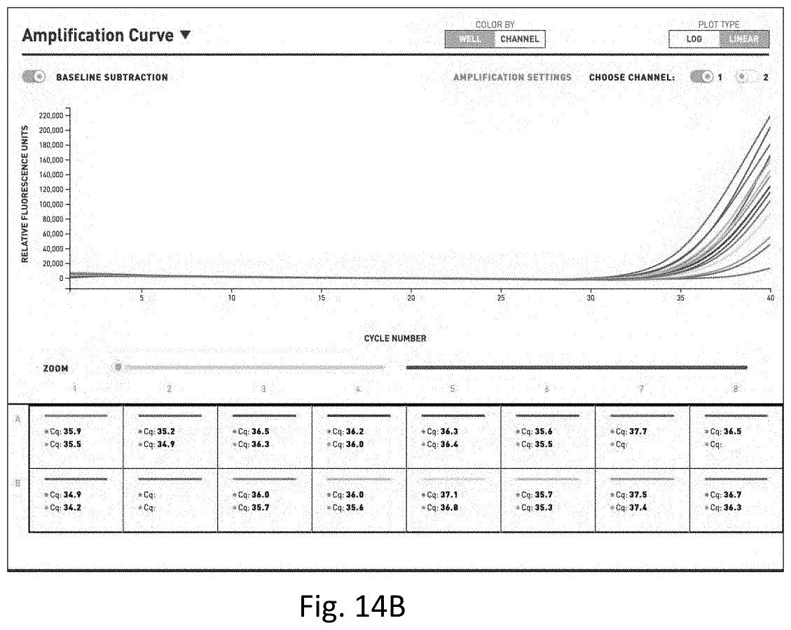

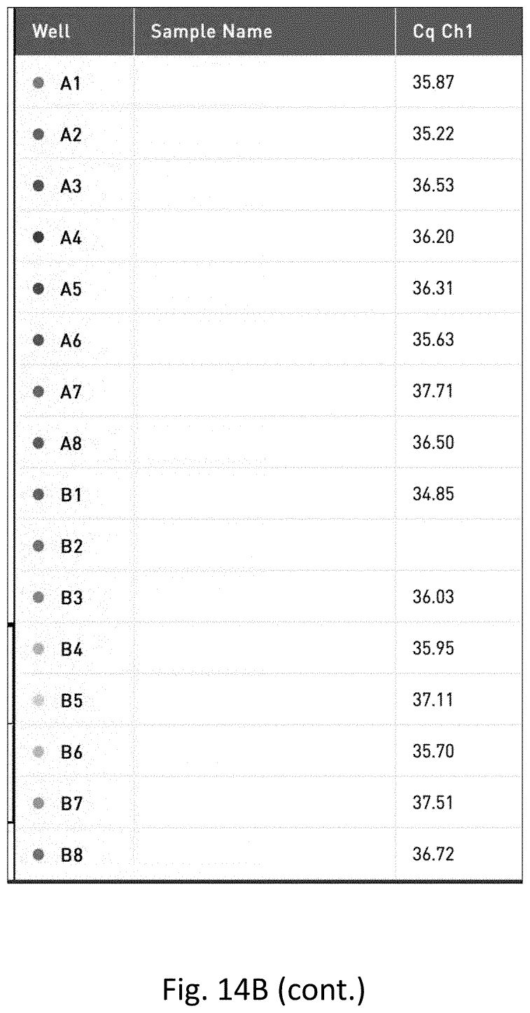

As shown in FIG. 14, detection and quantification of very low levels of bacteria were only possible when the filter and concentration system were used.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A method for rapidly isolating and detecting waterborne pathogens within a liquid sample comprising the steps of:
filtering the liquid sample using a portable filtration device so that the waterborne pathogens forms a filtrate on a surface of a filter of the portable filtration device and purified liquid that results from the liquid sample being conducted across the filter is discharged from the portable filtration device;
using a fluid delivery device to introduce a lysing agent into the portable filtration device resulting in lysing of the waterborne pathogens and formation of a lysed waterborne pathogen solution;
using the fluid delivery device to remove an amount of the lysed waterborne pathogen solution; and
introducing at least a portion of the removed amount of lysed waterborne pathogen solution into a molecular detection device that is configured to detect whether target waterborne pathogens are present in the liquid sample.

2. The method of claim 1, wherein the step of filtering the liquid sample using the portable filtration device acts to concentrate the waterborne pathogens as the filtrate formed within the filter.

3. The method of claim 1, wherein the portable filtration device comprises a cartridge and the filter comprises a plurality of semi-permeable filtering elements; an inlet port for delivering the liquid sample into lumens of the plurality of semi-permeable filtering elements; an outlet port for discharging the purified liquid; and a vent port for venting air from the lumens of the plurality of semi-permeable filtering elements.

4. The method of claim 3, wherein the plurality of semi-permeable filtering elements comprises hollow fibers, with the inlet port being in fluid communication with first ends of the hollow fibers and the vent port being in fluid communication with second ends of the hollow fibers, the outlet port being in fluid communication with a hollow space surrounding the plurality of semi-permeable filtering elements.

5. The method of claim 3, wherein the step of filtering the liquid sample includes the step of pumping the liquid sample into the lumens of the plurality of semi-permeable filtering elements.

6. The method of claim 1, wherein the fluid delivery device comprises a syringe that is connected to the inlet and is operated by moving a plunger within a barrel in a first direction to deliver the lysing agent into the filter into contact with the residue such that the lysing agent absorbs the residue and forms the lysed waterborne pathogen solution.

7. The method of claim 6, wherein the step of using the fluid delivery device to remove the amount of the lysed waterborne pathogen solution comprises moving the plunger within the barrel in a second direction to draw the amount of lysed waterborne pathogen solution into the barrel.

8. The method of claim 3, wherein the lysing agent is delivered into the lumens of the semi-permeable filtering elements.

9. The method of claim 8, wherein the lysing agent comprises a lysis buffer.

10. The method of claim 9, wherein a volume of the lysis buffer is greater than a total volume of lumens of the semi-permeable filtering elements.

11. The method of claim 1, wherein the portion of the removed amount of lysed waterborne pathogen solution comprises between 10 microliters and 50 microliters of solution.

12. The method of claim 1, wherein the portion of the removed amount of lysed waterborne pathogen solution is delivered into the one or more test wells of the molecular detection device, each well containing lyophilized primers, cap oligos, probes and master mix for polymerase chain reaction detection and are reconstituted with another solution.

13. The method of claim 1, wherein the molecular detection device is configured to incubate the lysed waterborne pathogen solution under amplification conditions with oligonucleotide primers and DNA polymerase and is configured to detect amplified target DNA to determine the presence or absence in the liquid of target pathogens or indicator microorganisms carrying selected target DNA nucleotide sequence.

14. The method of claim 1, wherein the liquid sample has a volume between about five-hundred milliliters to about 100 gallons.

15. The method of claim 1, wherein the liquid sample has a volume of about one liter.

16. A method for rapidly isolating and detecting waterborne pathogens within a liquid sample comprising the steps of:
filtering the liquid sample using a portable filtration device so that the waterborne pathogens forms a concentrated filtrate that comprises the waterborne pathogens and is contained within the portable filtration device;
introducing a lysing agent into the portable filtration device resulting in lysing of the waterborne pathogens and formation of a lysed waterborne pathogen solution that is contained within the portable filtration device;
removing an amount of the lysed waterborne pathogen solution from the portable filtration device; and
introducing at least a portion of the removed amount of lysed waterborne pathogen solution into a molecular detection device that is configured to detect whether target waterborne pathogens are present in the liquid sample.

17. A portable system for rapidly detecting waterborne pathogens comprising:
a portable filtration device including a first port for receiving a liquid sample to be tested; a second port for venting air and a third port for discharging purified liquid, the portable filtration device including a plurality of semi-permeable filtering elements for filtering the liquid sample and generating a filtrate within the semi-permeable filtering elements, the filtrate containing the waterborne pathogens;
a delivery device configured for being sealingly mated to the first port and configured to deliver a lysing agent into lumens of the plurality of semi-permeable filtering elements for lysing of the waterborne pathogens and formation of a lysed waterborne pathogen solution; and
a molecular detection device for analyzing the lysed waterborne pathogen solution and detecting whether target waterborne pathogens are present in the liquid sample.

18. The system of claim 17, wherein the plurality of filtering elements comprises a plurality of hollow fibers having a pore size from about 0.002 micron to about 0.01 micron.

19. The system of claim 17, wherein the portable filtration device has a filtration capacity to reduce the volume of the liquid by a factor of at least 10E-5.

20. The system of claim 17, wherein the molecular detection device includes test wells and the molecular detection device is configured to incubate the lysed waterborne pathogen solution under amplification conditions with oligonucleotide primers and DNA polymerase; and detect amplified target DNA to determine the presence or absence in the liquid sample of target pathogens or indicator microorganisms carrying the selected target DNA nucleotide sequence.

21. A kit for rapidly detecting waterborne pathogens comprising:
a portable filtration device including a first port for receiving a liquid sample to be tested; a second port for venting air and a third port for discharging purified liquid, the portable filtration device including a plurality of hollow semi-permeable fibers for filtering the liquid sample and generating a filtrate within lumens of the hollow semi-permeable fibers, the filtrate containing the waterborne pathogens in a concentrated form;
a syringe configured for being sealingly mated to the first port and configured to deliver a lysing agent into lumens of the plurality of hollow semi-permeable fibers for lysing of the waterborne pathogens and formation of a lysed waterborne pathogen solution, the syringe further configured for removing the lysed waterborne pathogen solution from within the lumens; and
a molecular detection device for analyzing the lysed waterborne pathogen solution and detecting whether target waterborne pathogens are present in the liquid sample.
